(19) United States
(12) Patent Application Publication
(10) Pub. No.: US 2014/0171862 A1
(43) Pub. Date: Jun. 19, 2014

(54) MULTI-CHAMBER SYRINGE

(71) Applicant: Manfred Weidner, Bensheim (DE)

(72) Inventor: Manfred Weidner, Bensheim (DE)

(73) Assignee: SANNER GMBH, Bensheim (DE)

(21) Appl. No.: 14/232,788

(22) PCT Filed: Jul. 3, 2012
(86) PCT No.: PCT/EP2012/002789

§ 371 Date: Jan. 14, 2014

(30) Foreign Application Priority Data
Jul. 15, 2011 (DE) 10 2011 107 764.6

(51) Int. Cl. A61M 5/19

(52) U.S. Cl. CPC A61M 5/19
USPC 604/85; 264/318

(57) ABSTRACT

A multi-chamber syringe with a syringe body having a wall which encloses a hollow space. The syringe body has a syringe outlet and an opening for receiving a piston displaceable in the hollow space, where at least one displaceable stopper is arranged in the hollow space and divides the hollow space into a first chamber, for receiving a first substance, and a second chamber, for receiving a second substance. The syringe body also has a channel through which the first substance can flow from the first chamber into the second chamber when the stopper is positioned in such a way that the channel connects the first chamber to the second chamber, where the channel is formed by a material cutout in the wall on a side of the wall facing toward the hollow space.

The invention relates to a multi-chamber syringe with a syringe body, of which the wall encloses a hollow space, wherein the syringe body has a syringe outlet and an opening for receiving a piston displaceable in the hollow space, wherein at least one displaceable stopper is arranged in the hollow space and divides the hollow space into a first chamber, for receiving a first substance, and a second chamber, for receiving a second substance, wherein the syringe body has a channel through which the first substance can flow from the first chamber into the second chamber when the stopper is positioned in such a way that the channel connects the first chamber to the second chamber.

A multi-chamber syringe of this kind is known from DE 10 2007 014 281 A1. Multi-chamber syringes are used in medicine for the administration of preparations that are composed of several substances. The substances can be present as liquids and also as solids. In a multi-chamber syringe designed as a twin-chamber syringe, two substances are located separate from each other in the syringe body prior to the administration, wherein both substances are separated by a stopper arranged in the syringe body. The second end of the syringe body is closed by a piston. The stopper prevents the two substances from being inadvertently mixed together beforehand. This is particularly advantageous if the substances are mutually incompatible or if the stability of the substances decreases when they are mixed.

The substances are mixed directly before being administered. For this purpose, the piston is pushed into the syringe body, as a result of which the stopper moves in the direction of the tip, on account of the incompressible medium located between piston and stopper. Approximately at its center, the syringe body has a bypass, which is formed by a bulging-out of the wall of the syringe body. When the stopper reaches the bypass, the substance located between stopper and piston flows into the hollow space located in front of the stopper and mixes with the substance that is located there. Upon completion of the mixing process, and after the air located in the hollow space has been removed, the multi-component preparation can be administered.

In the subject matter known from DE 10 2007 014 281 A1, a disadvantage is that the production of the syringe body requires a number of steps and is therefore expensive.

The object of the invention is to develop a multi-chamber syringe which can be produced easily, in particular from the point of view of hygiene, and also at reasonable cost.

This object is achieved by the features of claims 1 and 13. Advantageous embodiments are set forth in the dependent claims.

To achieve said object, the channel is formed by a material cutout in the wall, specifically on that side of the wall facing toward the hollow space. The material cutout can be formed such that the external diameter is unaltered and extends continuously from the syringe outlet to the opening. In terms of its external diameter, the syringe body therefore has no abrupt change and instead extends smoothly and continuously. The material cutout is produced by primary forming. Here, the material cutout causes a reduction in the wall thickness across the whole area of the channel. In this embodiment, it is advantageous that the syringe body cannot be differentiated externally from a simple single-chamber syringe, even though a channel is formed in the inside wall, which channel functions as a bypass and can interconnect two chambers separated from each other by the stopper. This permits the use of an injection mold which, in respect of the die forming the outer wall, is identical to the die of an injection mold provided for a single-chamber syringe. The syringe outlet merges into a hollow tip, which is preferably designed in the form of a Luer cone or Luer lock. This embodiment allows conventional needles to be secured on the syringe outlet.

The piston and the stopper can be arranged in different positions in the hollow space of the syringe body. In a first position, namely the storage position, the stopper is located before the channel, as seen from the opening, and the syringe outlet is closed by a suitable seal, for example by a cap that sealingly surrounds the Luer cone. In the storage position, the piston is arranged in the hollow space in the area of the opening, wherein the piston and the stopper each bear sealingly on the inside wall of the syringe body. The piston and the stopper delimit a first chamber, while the stopper and the syringe outlet delimit a second chamber. In this position, the channel is arranged completely in the second chamber.

By pressure applied to the piston, the stopper moves in the direction of the syringe outlet on account of the substance located in the first chamber, until the stopper reaches the cross-over position in which it overlaps the channel. As soon as the stopper covers the channel, the channel connects the first chamber to the second chamber, and the substance located in the first chamber flows through the channel into the second chamber. The stopper remains in this position until all of the substance located in the first chamber has flowed into the second chamber. By pressure applied to the piston, the volume of the first chamber decreases, until the piston bears on the stopper.

In this position, namely the position of use, both media are completely mixed with each other and, if appropriate, the mixing process can be assisted by additionally shaking the multi-chamber syringe. By means of further pressure applied to the piston, the volume of the second chamber also decreases, such that the air possibly located in the second chamber flows out. Thereafter, the substances that have been mixed with each other can be administered.

The channel is preferably designed as a groove extending in the longitudinal direction of the syringe body. A groove designed in this way is particularly easy to produce. In this context, it is conceivable that the groove is formed in the syringe body following the primary forming of said syringe body. However, it is particularly preferable that the groove is already generated during the primary forming, since this method of production allows a reduction in the number of production steps. The groove is designed such that its longitudinal extent is greater than the longitudinal extent, or thickness, of the stopper. In this way, it is ensured that the channel connects the two chambers to each other when the stopper covers the channel. It is also conceivable to provide a plurality of channels, which are distributed about the inner circumference of the syringe body. In this embodiment, quicker cross-over is permitted as a result of the increased volumetric flow.

The channel can have a first portion, in which the groove base of the groove extends obliquely with respect to the center axis of the syringe body. As a result of the obliquely extending portion, the cross section of the channel decreases continuously which, particularly in the inflow area of the channel, leads to an improvement in the flow conditions during the cross-over. Moreover, the obliquely extending portion permits easy production of the syringe body, particularly by improving the removability of the core belonging to the injection mold.

The channel can have a second portion, in which the groove base extends parallel to the center axis of the syringe body. This portion is preferably assigned to the region in which the stopper covers the channel in the cross-over position. In this region, a constant cross section is obtained without flow obstruction.

The first portion is preferably located nearer to the opening, and the second portion is located nearer to the syringe outlet. It is advantageous that the flow conditions thus improve during the inward flow of the substance into the channel. By contrast, the second portion in the region of the second chamber can open into an edge, such that the substance flowing across is diverted and, in this way, the mixing process with the substance located in the second chamber is improved.

Starting from the syringe outlet, the internal diameter of the syringe body can widen in the direction of the opening. This results in the syringe body having an inside wall that is designed conically at least in some regions. This shape improves the removability of the core from the injection mold.

For the widening of the internal diameter, the inside wall of the syringe body extends at an opening angle with respect to the center axis. The widening of the internal diameter can take place in regions, wherein in particular the opening angle of the first region assigned to the channel can be greater than the second region assigned to the syringe outlet and/or the third region assigned to the opening. The conical bevels formed by the enlargement of the internal diameter are also called drafts. These improve the removability of the core belonging to the injection mold. If the channel is produced in one go with the primary forming of the syringe body, a tool is needed for shaping the channel, which tool is preferably formed from the core. However, this tool may have sharp edges, which can cause scratches in the inside wall of the syringe body during removal from the mold. As a result of the larger draft arranged in the region of the channel, or of the cross-sectional widening extending at a greater angle, the core and the tool for shaping the channel are already free after a short distance during the removal from the mold, and they no longer have any contact with the inside wall of the syringe body. Formation of scratches on the inside wall is thereby avoided.

Moreover, as a result of the diameter decreasing in the direction of the syringe outlet, the contact pressure on the stopper guided in the syringe body increases while said stopper is moving from the storage position to the cross-over position. This also ensures that the stopper remains in the cross-over position while the substance flows across.

The opening of the syringe body can be assigned an annular grip plate. The grip plate serves as a grip during the administration of the fluids and permits targeted control of the piston. On account of the annular design, the syringe body is easier to handle during mechanical filling of the multi-chamber syringe.

The syringe body is preferably made from a plastics injection molding material, in particular from cyclo-olefin copolymer. Cyclo-olefin copolymer is an amorphous and therefore transparent injection-moldable olefin. Moreover, in addition to being very stiff and hard, the material also has good biocompatibility.

The stopper and the piston are preferably made from an elastomer material, in particular from bromobutyl rubber (BIIR). Bromobutyl is a halogen-modified isobutene-isoprene rubber from the group of synthetic elastomers. Bromobutyl is distinguished by good resistance to acids and bases and by very low gas permeability. Substances can therefore be stored in the multi-chamber syringe over a long period of time.

The stopper and the piston can be provided with circumferential ribs. The circumferential ribs improve the sealing action of the stopper and the piston, without impairing the sliding behavior. To improve the sliding behavior and to avoid stick-slip, it is possible for the stopper, the piston and the inside wall of the syringe body to be provided with a coating, preferably a silicone-based coating.

The syringe body, including the channel formed in its wall, is preferably produced by an injection molding method. It is advantageous here that the syringe body and the channel are produced in one go, without the need for additional downstream manufacturing steps. The manufacture of the multi-chamber syringe is thereby simplified, and the production costs are reduced.

In the method according to the invention for producing a multi-chamber syringe as claimed in one of the preceding claims, the syringe body is formed by means of plastics injection molding, wherein the channel, during the injection molding operation, is generated at the same time as the primary forming of the syringe body. By means of a suitable tool, which is operatively connected to the injection-mold core forming the inside wall of the syringe body, integrated production of the syringe body and the channel is possible.

For the primary forming of the syringe body and the simultaneous production of the channel, an oblique slide can protrude from the core forming the inside wall of the syringe body and thus forms, on the inside wall of the syringe body, the channel jutting into the wall, wherein the oblique slide is drawn into the core when the injection molding operation is completed, and the core is thereafter withdrawn from the hollow space of the syringe body in the direction of the opening. The oblique slide is particularly advantageous in terms of its small dimensions. As a result of the small dimensions, cooling elements can be integrated in the core and can protrude into the area of the syringe outlet.

A number of embodiments of the multi-chamber syringe according to the invention are explained in more detail below with reference to the schematic figures, in which:

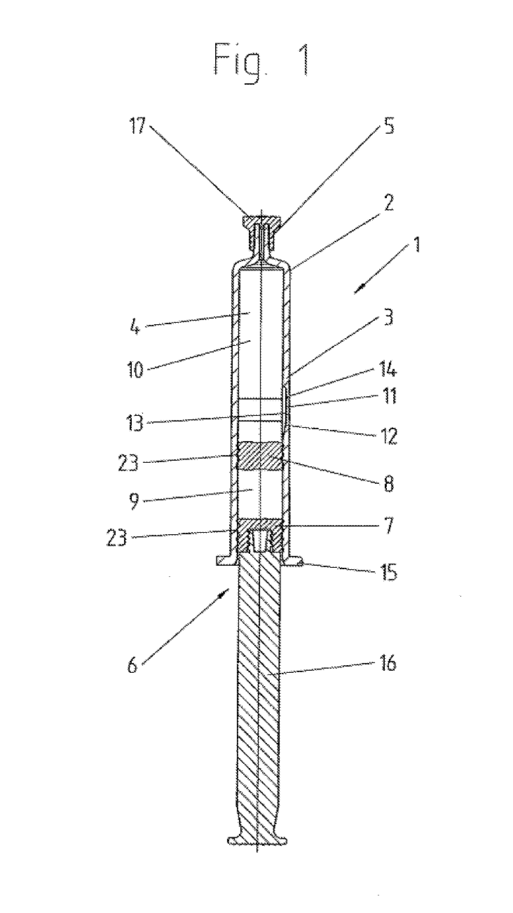

FIG. 1 shows the multi-chamber syringe in section, in the storage position;

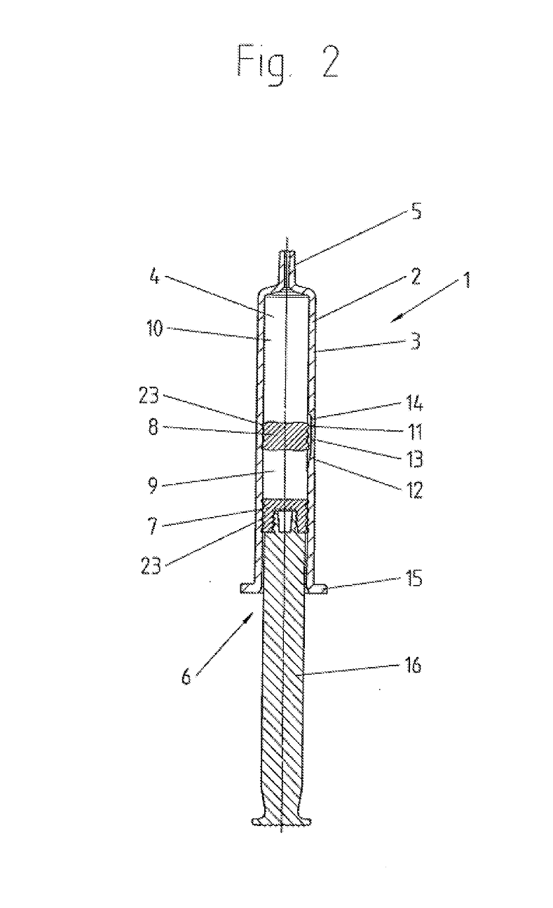

FIG. 2 shows the multi-chamber syringe in section, in the cross-over position;

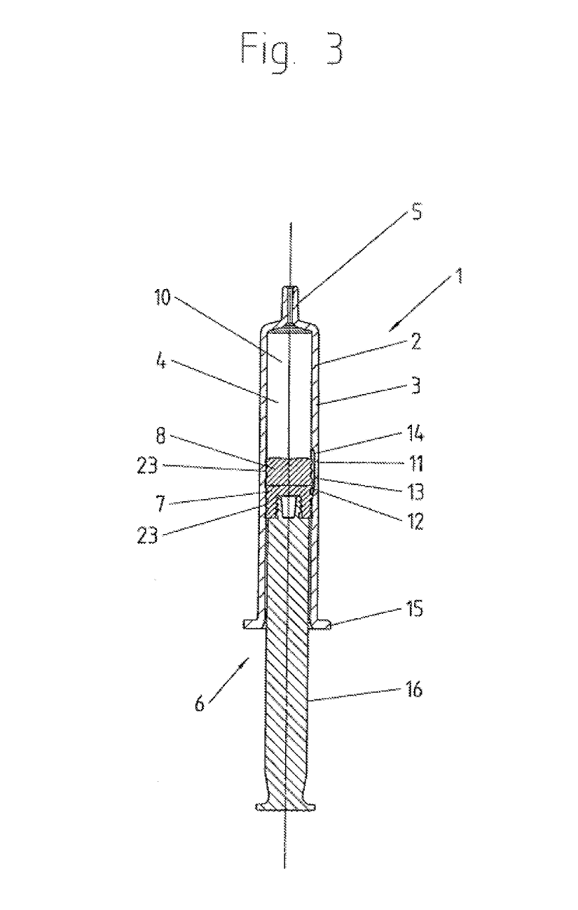

FIG. 3 shows the multi-chamber syringe in section, in the position of use;

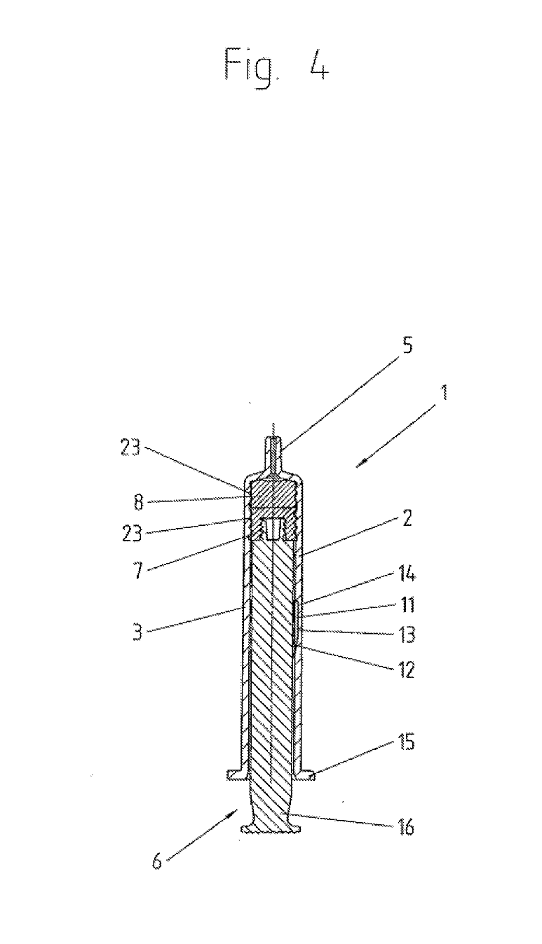

FIG. 4 shows the multi-chamber syringe in section, when empty;

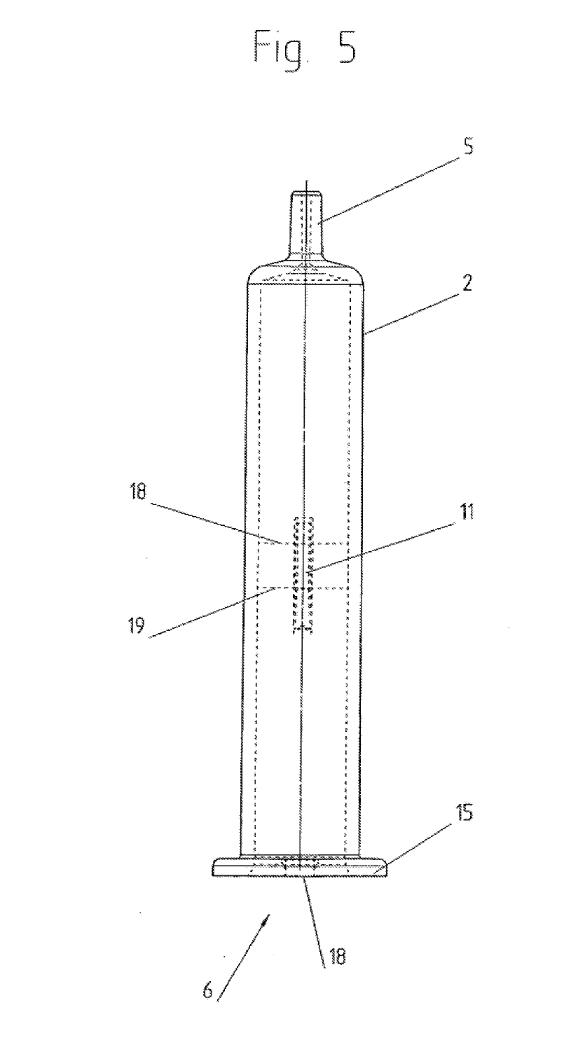

FIG. 5 shows the syringe body in a longitudinal view;

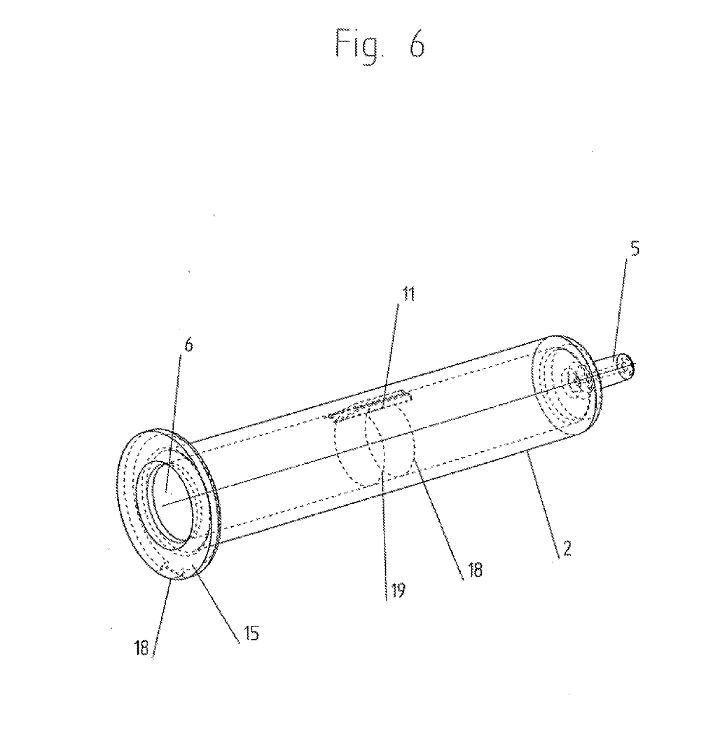

FIG. 6 shows the syringe body in a perspective view;

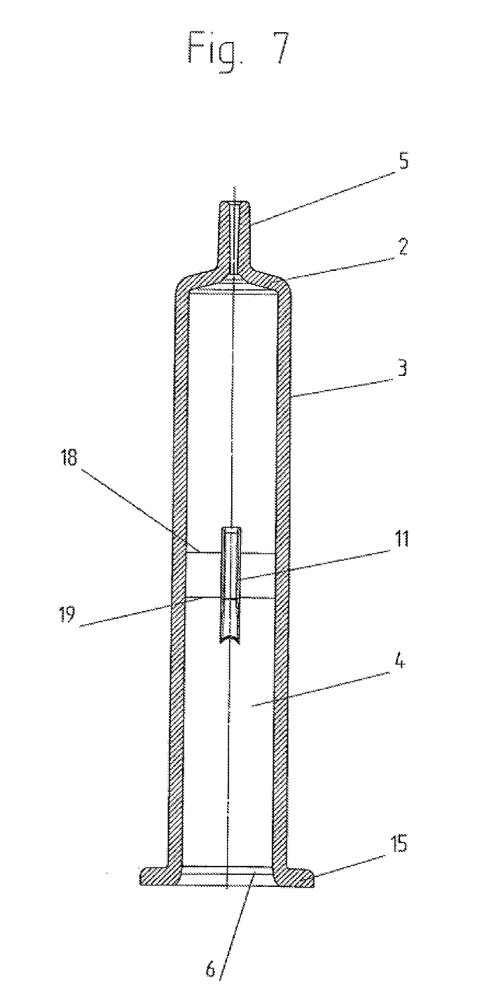

FIG. 7 shows the syringe body in longitudinal section;

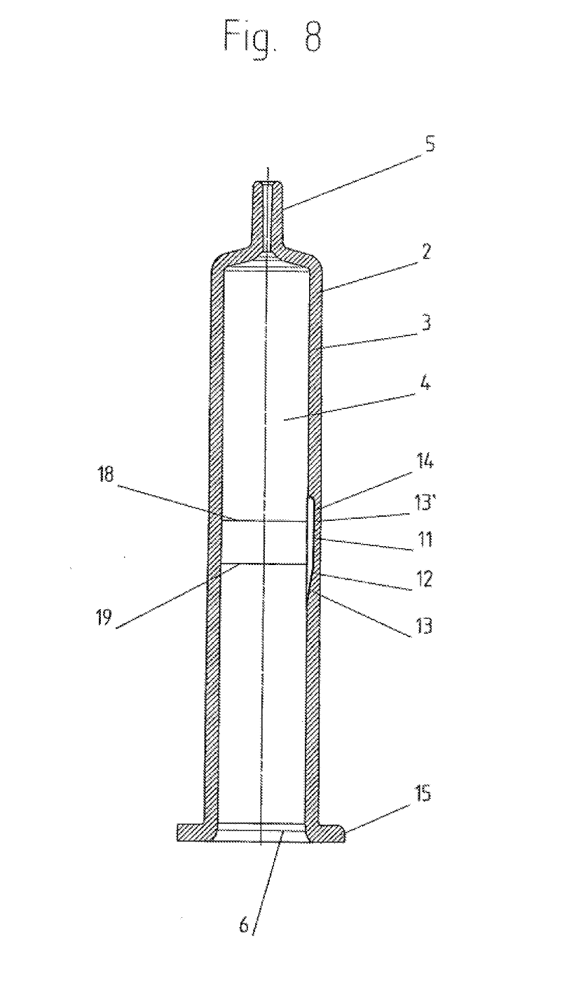

FIG. 8 shows the syringe body in longitudinal section;

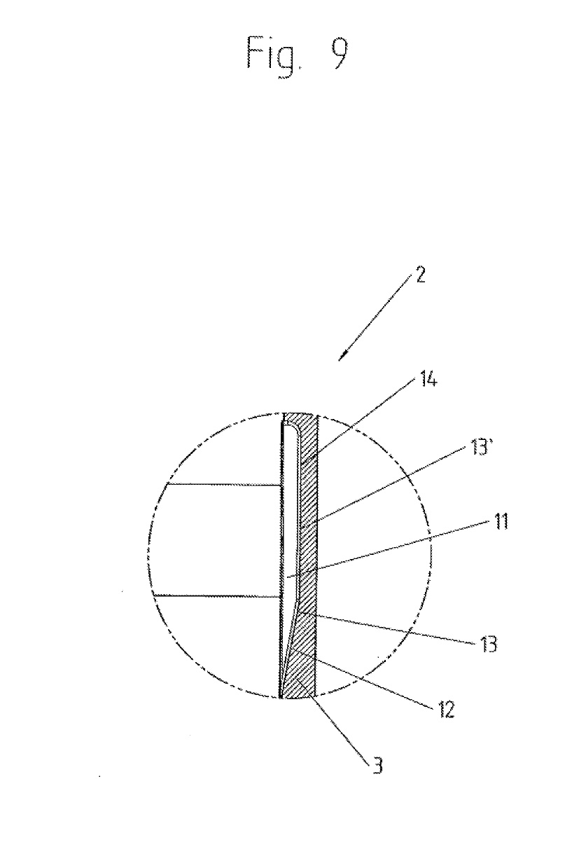

FIG. 9 shows, in detail, the channel in longitudinal section;

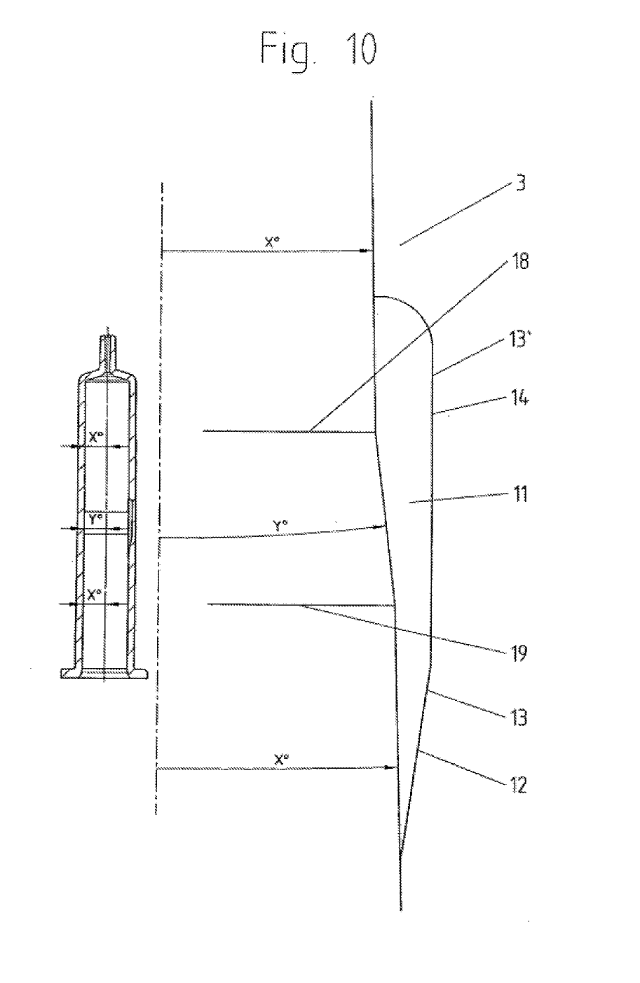

FIG. 10 shows a schematic view of the widening of the cross section of the inside wall;

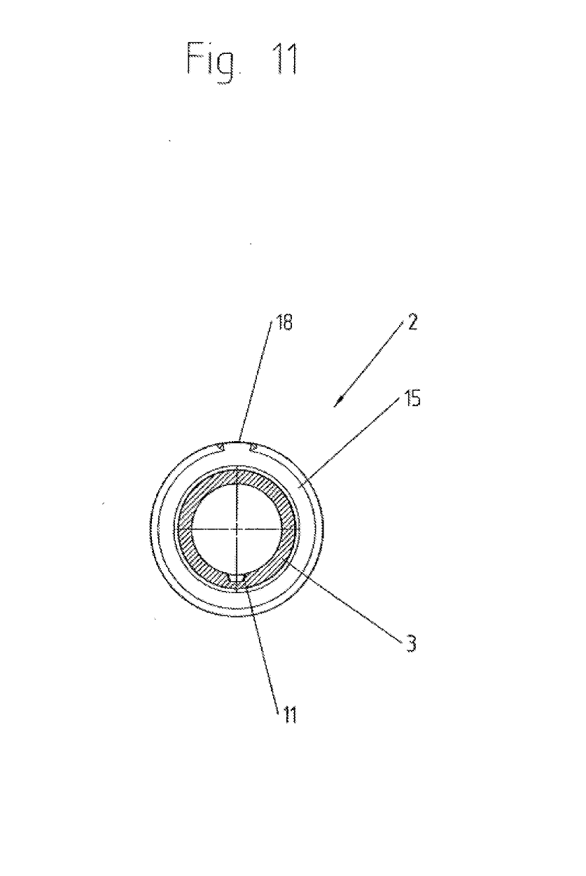

FIG. 11 shows the syringe body in cross section;

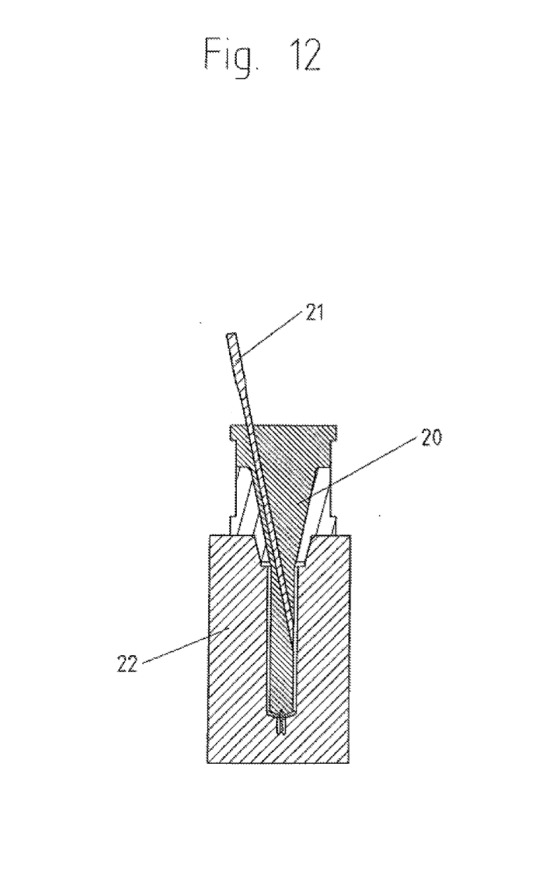

FIG. 12 shows the injection mold in the primary forming operation;

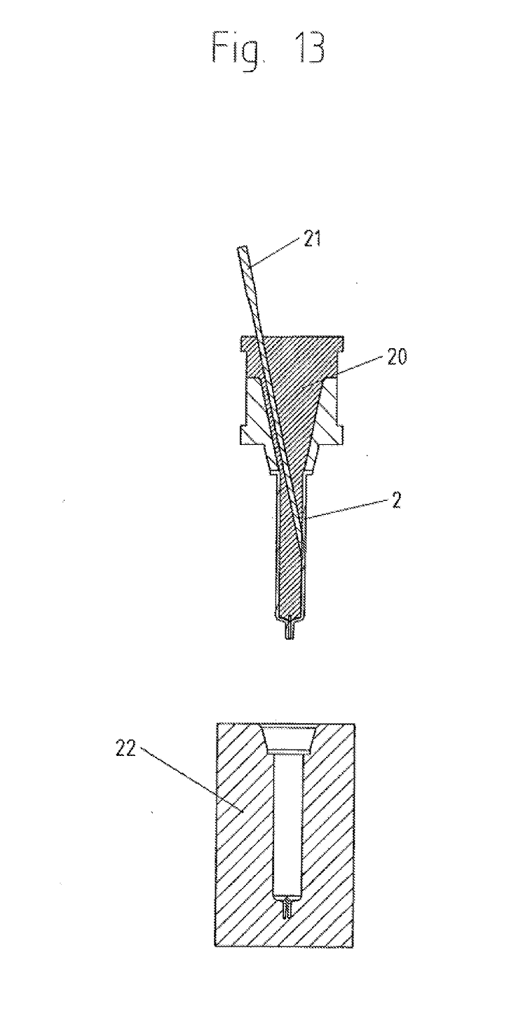

FIG. 13 shows the syringe body partially removed from the injection mold;

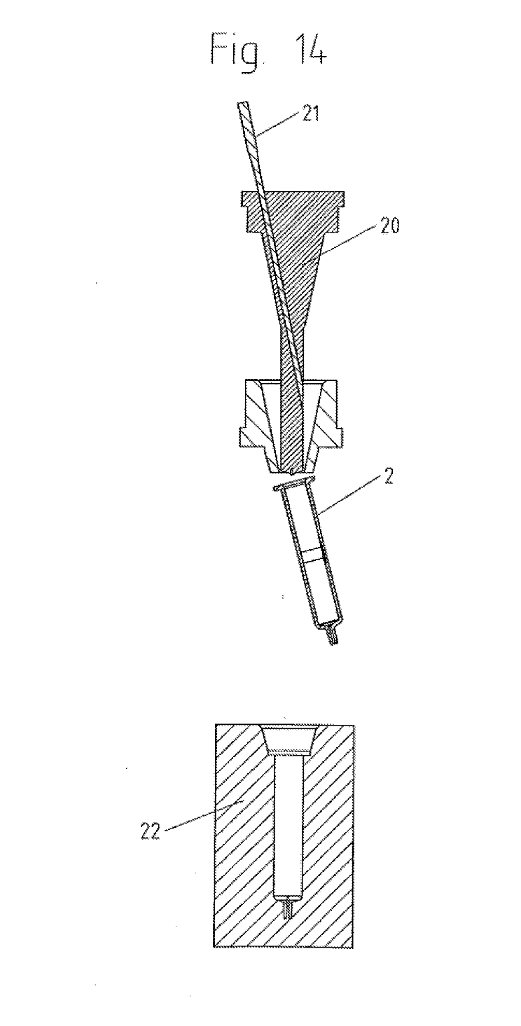

FIG. 14 shows the syringe body completely removed from the injection mold.

FIG. 1 shows a multi-chamber syringe 1 with a syringe body 2, of which the wall 3 encloses a hollow space 4. The syringe body 2 is designed as an injection-molded part and is made of a transparent, translucent and injection-moldable plastic having sufficient hardness. A material of this kind is, for example, cyclo-olefin copolymer (COC). The syringe body 2 has a syringe outlet 5, which is shaped as a tip and tapers conically in the direction of the free end. Preferably, the syringe outlet 5 has the form of a Luer cone. In another embodiment, the syringe outlet 5 can also have a thread in the form of a Luer lock. The syringe outlet 5 is closed by a cap 17 made of elastic plastic, preferably of bromobutyl (BIIR).

The syringe body has an opening 6 for receiving a piston 7 displaceable in the hollow space 4, wherein at least one displaceable stopper 8 is arranged in the hollow space 4 and divides the hollow space 4 into a first chamber 9, for receiving a first substance, and a second chamber 10, for receiving a second substance. The substances can be present as liquids and also as powders. The volume of the second chamber 10 is dimensioned such that the second chamber 10 can receive both the substance of the first chamber 9 and also the substance of the second chamber 10, plus an added volume for improving the mixing process. The piston 7 and the stopper 8 are made from an elastomer material, preferably from bromobutyl rubber (BIIR). The stopper 8 and the piston 7 are provided with circumferential ribs 23 in order to improve the sealing action.

For connecting first chamber 9 and second chamber 10, the syringe body 2 has a channel 11 through which the first substance can flow from the first chamber 9 into the second chamber 10 when the stopper 8 is positioned in such a way that the channel 11 connects the first chamber 9 to the second chamber 10. According to the invention, the channel 11 is formed by a material cutout in the wall 3, specifically on that side of the wall 3 facing toward the hollow space 4. The piston 7 is provided with an internal thread, into which a piston rod 16 is screwed.

The piston 7 and the stopper 8 can be arranged in different positions in the hollow space 4 of the syringe body 2. In a first position, namely the storage position, the stopper 8 is located before the channel 11, as seen from the opening 6, and the syringe outlet 5 is closed by a cap 17. In the storage position, the piston 7 is arranged in the hollow space 4 in the area of the opening 6, wherein the piston 7 and the stopper 8 each bear sealingly on the inside wall of the syringe body 2. The piston 7 and the stopper 8 delimit the first chamber 9, while the stopper 8 and the syringe outlet 2 delimit the second chamber 10. In this position, the channel 11 is arranged completely in the second chamber 10.

After removal of the cap 17, and by pressure applied to the piston 7, the stopper 8 moves in the direction of the syringe outlet 5 on account of the mostly incompressible substance located in the first chamber 9, until the stopper 8 reaches the cross-over position in which it overlaps the channel 11. This position is shown in FIG. 2.

As soon as the stopper 8 covers the channel 11, the channel 11 connects the first chamber 9 to the second chamber 10, and the substance located in the first chamber 9 flows through the channel 11 into the second chamber 10. The stopper 8 remains in this position until all of the substance located in the first chamber 9 has flowed into the second chamber 10. For this purpose, the contact pressure of the stopper 8 against the inside wall of the syringe body 2 is such that the force needed to move the stopper 8 in the syringe body 2 is greater than the resistance that is encountered by the substance flowing through the channel 11. By pressure applied to the piston 7, the volume of the first chamber 9 decreases, until the piston 7 bears on the stopper 8. This position is shown in FIG. 3.

In this position, namely the position of use, both substances are mixed with each other and, by means of further pressure applied to the piston 7, the volume of the second chamber 10 also decreases, such that the air possibly located in the second chamber 10 flows out. Thereafter, the substances that have been mixed with each other can be administered, once a needle has been fitted onto the syringe outlet 5.

FIG. 4 shows the position of the piston 7 and the stopper 8 after administration of the two substances that have been mixed with each other.

FIG. 5 shows a longitudinal view of the syringe body 2 of the above-described multi-chamber syringe 1. In this view, the elements arranged in the hollow space 4 of the syringe body 2, for example the channel 11, are shown by dashed lines.

FIG. 6 shows the same syringe body in a perspective view. It will be noted that the opening 6 of the syringe body 2 is assigned a grip plate 15, which is of annular shape.

FIG. 7 shows the above-described syringe body 2 in longitudinal section. The plan view shows the channel 11, which is designed as a groove extending in the longitudinal direction of the syringe body 2. The channel 11 is formed out of the wall 3 of the syringe body 2, as a result of which the wall 3 has a reduced thickness in this area.

The above-described syringe body is shown once again in longitudinal section in FIG. 8, the channel 11 likewise being shown in section in this view. It will be seen from this that the channel 11 has a first portion 12, in which the groove base 13 of the groove extends obliquely with respect to the center axis of the syringe body 2, and a second portion 14, in which the groove base 13′ extends parallel to the center axis of the syringe body 2. The first portion 12 is located nearer to the opening 6, and the second portion 14 is located nearer to the syringe outlet 5.

FIG. 9 shows a detail of the channel 11 described in FIG. 8.

Starting from the syringe outlet 5, the internal diameter of the syringe body 2 widens in the direction of the opening 6. However, the widening of the internal diameter does not extend continuously but instead in regions. The inside wall of the syringe body 2 extends at an opening angle with respect to the center axis. The widening of the internal diameter takes place in regions, wherein the opening angle of the first region assigned to the channel 11 is greater than the second region assigned to the syringe outlet 5 and greater than the third region assigned to the opening 6. The second region begins at the syringe outlet and ends at the first line 18, which can be seen in the area of the channel 11. The first region, namely the region assigned to the channel, is delimited by the first line 18 and the second line 19. The third region begins at the second line 19 and ends at the opening 6. The opening angle of the second region and the opening angle of the third region are equal, and the opening angle of the first region is greater than the opening angle of the second region and of the third region.

This gradation of the opening angles is shown schematically in FIG. 10.

FIG. 11 shows the above-described syringe body 2 in cross section. A gate region 18 is arranged in the area of the grip plate 15 of the syringe body 2. The material of the syringe body 2 is delivered in this region during the injection molding operation.

FIGS. 12 to 14 show a production method and a mold for producing a syringe body 2 for a multi-chamber syringe 1 as described above. The syringe body 2 is produced in an injection molding method, wherein the channel 11, during the injection molding operation, is generated at the same time as the primary forming of the syringe body 2. For this purpose, prior to the delivery of the material, an oblique slide 21 is extended out from the core 20 forming the inside wall of the syringe body 2, which oblique slide 21 thus forms, on the inside wall of the syringe body 2 forming upon delivery of the plastic, the channel 11 jutting into the wall 3. The oblique slide 21 is drawn into the core 20 when the injection molding operation is completed, and the core 20 and syringe body 2 are removed from the injection mold 22, as can be seen in FIG. 14. The core 20 is thereafter withdrawn from the hollow space 4 of the syringe body 2 in the direction of the opening 6.

1. A multi-chamber syringe including a syringe body having a wall that encloses a hollow space wherein the syringe body has a syringe outlet and an opening for receiving a piston displaceable in the hollow space, where at least one displaceable stopper is arranged in the hollow space and divides the hollow space into a first chamber, for receiving a first substance, and a second chamber, for receiving a second substance, wherein the syringe body has a channel through which the first substance can flow from the first chamber into the second chamber when the stopper is positioned in such a way that the channel connects the first chamber to the second chamber wherein the channel is formed by a material cutout in the wall on a side of the wall facing toward the hollow space.

2. The multi-chamber syringe as claimed in claim 1, wherein the channel is designed as a groove extending in the longitudinal direction of the syringe body.

3. The multi-chamber syringe as claimed in claim 2, wherein the channel has a first portion, in which the groove base of the groove extends obliquely with respect to the center axis of the syringe body.

4. The multi-chamber syringe as claimed in one of claim 3, wherein the channel has a second portion, in which the groove base extends parallel to the center axis of the syringe body.

5. The multi-chamber syringe as claimed in claim 4, wherein the first portion is located nearer to the opening, and the second portion is located nearer to the syringe outlet.

6. The multi-chamber syringe as claimed in claim 1, wherein starting from the syringe outlet, an internal diameter of the syringe body widens in the direction of the opening.

7. The multi-chamber syringe as claimed in claim 6, wherein for the widening of the internal diameter, the inside wall of the syringe body extends at an opening angle with respect to the center axis, wherein the widening of the internal diameter takes place in regions, and wherein an opening angle of a first region assigned to the channel is greater than a second region assigned to the syringe outlet and a third region assigned to the opening.

8. The multi-chamber syringe as claimed in claim 1, wherein the opening of the syringe body is assigned an annular grip plate.

9. The multi-chamber syringe as claimed in claim 1, wherein the syringe body is made from cyclo-olefin copolymer.

10. The multi-chamber syringe as claimed in claim 1, wherein the stopper and the piston are made from bromobutyl rubber.

11. The multi-chamber syringe as claimed in claim 1, wherein the stopper and the piston include circumferential ribs.

12. The multi-chamber syringe as claimed in claim 1, wherein the syringe body including the channel formed in its wall, is produced by an injection molding method.

13. A method for producing a multi-chamber syringe as claimed in claim 1, wherein the syringe body is formed by means of plastics injection molding, wherein the channel, during the injection molding operation, is generated at the same time as the primary forming of the syringe body.

14. The method as claimed in claim 13, wherein for the primary forming of the syringe body and the simultaneous production of the channel, an oblique slide protrudes from the core forming the inside wall of the syringe body and thus forms, on the inside wall of the syringe body, the channel jutting into the wall, wherein the oblique slide is drawn into the core when the injection molding operation is completed, and the core is thereafter withdrawn from the hollow space of the syringe body in the direction of the opening.
